(19) United States
(12) Patent Application Publication
(10) Pub. No.: US 2009/0170848 A1
(43) Pub. Date: Jul. 2, 2009

(54) THIAZOLONES FOR USE AS PI3 KINASE INHIBITORS

(75) Inventors: Dahsyant Dhanak, Collegeville, PA (US); Steven David Knight, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION, Philadelphia, PA (US)

(21) Appl. No.: 12/281,183

(22) PCT Filed: Mar. 2, 2007
(86) PCT No.: PCT/US07/63114

§ 371 Date: Nov. 13, 2008

Related U.S. Application Data
(60) Provisional application No. 60/778,585, filed on Mar. 2, 2006.

(51) Int. Cl. A61K 31/5377; A61K 31/498; A61K 31/4709; A61P 35/00

(52) U.S. Cl. 514/234.8; 514/249; 514/314

(57) ABSTRACT

Invented is a method of inhibiting the activity/function of PI3 kinases using substituted thiazolones. Also invented is a method of treating one or more disease states selected from: autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries by the administration of substituted thiazolones.

### FIELD OF THE INVENTION

This invention relates to the use of substituted thiazolones for the modulation, notably the inhibition of the activity or function of the phosphor-inositide-3′OH kinase family (hereinafter PI3 kinases), suitably, PI3Kα, PI3Kδ, PI3Kβ, and/or PI3Kγ. Suitably, the present invention relates to the use of substituted thiazolones in the treatment of one or more disease states selected from: autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries.

### BACKGROUND OF THE INVENTION

Cellular plasma membranes can be viewed as a large store of second messenger that can be enlisted in a variety of signal transduction pathways. As regards function and regulation of effector enzymes in phospholipids signaling pathways, these enzymes generate second messengers from the membrane phospholipids pool (class I PI3 kinases (e.g. PI3Kgamma)) are dual-specific kinase enzymes, means they display both: lipid kinase (phosphorylation of phosphor-inositides) as well as protein kinase activity, shown to be capable of phosphorylation of other protein as substrates, including auto-phosphorylation as intramolecular regulatory mechanism. These enzymes of phospholipids signaling are activated in response to a variety of extra-cellular signals such as growth factors, mitogens, integrins (cell-cell interactions) hormones, cytokines, viruses and neurotransmitters such as described in Scheme 1 hereinafter and also by intra-cellular cross regulation by other signaling molecules (cross-talk, where the original signal can activate some parallel pathways that in a second step transmit signals to PI3Ks by intra-cellular signaling events), such as small GTPases, kinases or phosphatases for example. The inositol phospholipids (phosphoinositides) intracellular signaling pathway begins with binding of a signaling molecule (extra cellular ligands, stimuli, receptor dimerization, transactivation by heterologous receptor (e.g. receptor tyrosine kinase)) to a G-protein linked transmembrane receptor integrated into the plasma membrane.

PI3K converts the membrane phospholipids PIP(4,5)2 into PIP(3,4,5)3 which in turn can be further converted into another 3′ phosphorylated form of phosphoinositides by 5′-specific phosphor-inositide phophatases, thus PI3K enzymatic activity results either directly or indirectly in the generation of two 3′-phosphoinositide subtypes that function as 2^{nd} messengers in intr-cellular signal transduction (Trends Biochem. Sci. 22(7) p.267-72 (1997) by Vanhaesebroeck et al.: Chem. Rev. 101(8) p.2365-80 (2001) by Leslie et al (2001); Annu. Rev. Cell. Dev. Biol. 17p, 615-75 (2001) by Katso et al. and Cell. Mol. Life Sci. 59(5) p.761-79 (2002) by Toker et al.). Multiple PI3K insoforms categorized by their catalytic subunits, their regulation by corresponding regulatory subunits, expression patterns and signaling-specific functions (p110α, β, and γ) perform this enzymatic reaction (Exp. Cell. Res. 25 (1) p. 239-54 (1999) by Vanhaesebroeck and Katso et al., 2001, above).

The evolutionary conserved insoforms p110α and β are ubiquitously express, which δ and γ are more specifically expressed in the haematopoietic cell system, smooth muscle cells, myocytes and endothelial cells (Trends Biochem. Sci. 22(7) p.267-72 (1997) by Vanhaesebroeck et al.). Their expression might also be regulated in an inducible manner depending on the cellular, tissue type and stimuli as well as disease context.

To date, eight mammalian PI3Ks have been identified, divided into three main classes (I, II, and III) on the basis of sequence homology, structure, binding partners, mode of activation, and substrate preference in vitro. Class I PI3Ks can phosphorylate phosphatidylinositol (PI), phosphatidylinositol-4-phosphate,m and phosphatidylinositol-4,5-biphosphate (PIP2) to produce phosphatidylinositol-3-phosphate (PIP), phosphatidylinositol-3,4-biphosphate, and phosphatidylinositol-3,4,5-triphosphate, respectively. Class II PI3Ks phosphorylate PI and phosphatidylinositol-4-phosphate. Class III PI3Ks can only phosphorylate PI (Vanhaesebrokeck et al., 1997, above; Vanhaesebroeck et al., 1999, above and Leslie et al, 2001, above) G-protein coupled receptors mediated phosphoinositide 3′OH-kinase activation via small GTPases such as Gβγ and Ras, and consequently PI3K signaling plays a central role in establishing and coordinating cell polarity and dynamic organization of the cytoskeleton—which together provides the driving force of cells to move.

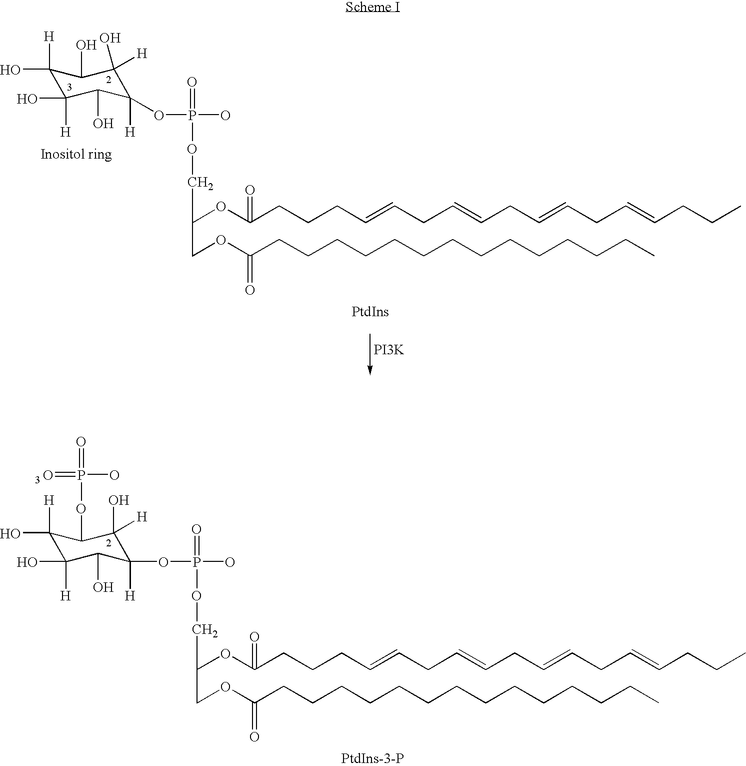

As illustrated in Scheme 1 above, Phosphoinositide 3-kinase (PI3K) is involved in the phosphorylation of Phosphatidylinositol (PtdIns) on the third carbon of the inositol ring. The phosphorylation of PtdIns to 3,4,5-triphosphate (PtdIns(3,4,5)P₃), PtdIns(3,4)P₂ and PtdIns(3)P acts as second messengers for a variety of signal transduction pathways, including those essential to cell proliferation, cell differentiation, cell growth, cell size, cell survival, apoptosis, adhesion, cell motility, cell migration, chemotaxis, invasion, cytoskeletal rearrangement, cell shape changes, vesicle trafficking and metabolic pathway (Katso et al., 2001, above and Mol. Med. Today 6(9) p. 347-57 (2000) by Stein). Chemotaxis—the directed movement of cells toward a concentration gradient of chemical attractants, also called chemokines is involved in many important diseases such as inflammation/auto-immunity, neurodegeneration, antiogenesis, invasion/metastasis and wound healing (Immunol. Today 21(6) p. 260-4 (2000) by Wyman et al.; Science 287(5455) p. 1049-53 (2000) by Hirsch et al.; FASEB J. 15(11) p. 2019-21 (2001) by Hirsch et al. and Nat. Immunol. 2(2) p. 108-15 (2001) by Gerard et al.).

Recent advances using genetic approaches and pharmacological tools have provided insights into signalling and molecular pathways that mediate chemotaxis in response to chemoattractant activated G-protein coupled receptors PI3-Kinase, responsible for generating these phosphorylated signalling products, was originally identified as an activity associated with viral oncoproteins and growth factor receptor tyrosine kinases that phosphorylates phosphatidylinositol (PI) and its phosphorylated derivatives at the 3′-hydroxyl of the inositol ring (Panayotou et al., Trends Cell Biol. 2 p. 358-60 (1992)). However, more recent biochemical studies revealed that, class I PI3 kinases (e.g. class IB isoform PI3Kγ) are dual-specific kinase enzymes, means they display both: lipid kinase (phosphorylation of phospho-inositides) as well as protein kinase activity, shown to be capable of phosphorylation of other protein as substrates, including auto-phosphorylation as intra-molecular regulatory mechanism.

PI3-kinase activation, is therefore believe to be involved in a range of cellular responses including cell growth, differentiation, and apoptosis (Parker et al., Current Biology, 5 p. 577-99 (1995); Yao et al., Science, 267 p. 2003-05 (1995)). PI3-kinase appears to be involved in a number of aspects of leukocyte activation. A p85-associated PI3-kinase activity has been shown to physically associate with the cytoplasmic domain of CD28, which is an important costimulatory molecule for the activation of T-cells in response to antigen (Pages et al., Nature, 369 p. 327-29 (1994); Rudd, Immunity 4 p. 527-34 (1996)). Activation of T cells through CD28 lowers the threshold for activation by antigen and increases the magnitude and duration of the proliferative response. These effects are linked to increases in the transcription of a number of genes including interleukin-2 (IL2), an important T cell growth factor (Fraser et al., Science 251 p. 313-16 (1991)). Mutation of CD28 such that it can longer interact with PI3-kinase leads to a failure to initiate IL2 production, suggesting a critical role for PI3-kinase in T cell activation. PI3Kγ has been identified as a mediator of G beta-gamma-dependent regulation of JNK activity, and G beta-gamma are subunits of heterotrimeric G proteins (Lopez-Ilasaca et al., J. Biol. Chem. 273(5) p. 2505-8 (1998)). Cellular processes in which PI3Ks play an essential role include suppression of apoptosis, reorganization of the actin skeleton, cardiac myocyte growth, glycogen synthase stimulation by insulin, TNFα-mediated neutrophil priming and superoxide generation, and leukocyte migration and adhesion to endothelial cells.

Recently, (Laffargue et al., Immunity 16(3) p. 441-51 (2002)) it has been described that PI3Kγ relays inflammatory signals through various G(i)-coupled receptors and its central to mast cell function, stimuli in context of leukocytes, immunology includes cytokines, chemokines, adenosines, antibodies, integrins, aggregation factors, growth factors, viruses or hormones for example (J. Cell. Sci. 114(Pt 16) p. 2903-10 (2001) by Lawlor et al.; Laffargue et al., 2002, above and Curr. Opinion Cell Biol. 14(2) p. 203-13 (2002) by Stephens et al.).

Specific inhibitors against individual members of a family of enzymes provide invaluable tools for deciphering functions of each enzyme. Two compounds, LY294002 and wortmannin (cf. hereinafter), have been widely used as PI3-kinase inhibitors. These compounds are non-specific PI3K inhibitors, as they do not distinguish among the four members of Class I PI3-kinases. For example, the IC₅₀ values of wortmannin against each of the various Class I PI3-kinases are in the range of 1-10 nM. Similarly, the IC₅₀ values for LY294002 against each of these PI3-kinases is about 15-20 μM (Fruman et al., Ann. Rev. Biochem., 67, p. 481-507 (1998)), also 5-10 microM on CK2 protein kinase and some inhibitory activity on phospholipases. Wortmannin is a fungal metabolite which irreversibly inhibits PI3K activity by binding covalently to the catalytic domain of this enzyme. Inhibition of PI3K activity by wortmannin eliminates subsequent cellular response to the extracellular factor. For example, neutrophils respond to the chemokine fMet-Leu-Phe (fMLP) by stimulating PI3K and synthesizing PtdIns (3, 4, 5)P₃. This synthesis correlates with activation of the respirators burst involved in neutrophil destruction of invading microorganisms. Treatment of neutrophils with wortmannin prevents the fMLP-induced respiratory burst response (Thelen et al., Proc. Natl. Acad. Sci. USA, 91, p. 4960-64 (1994)). Indeed, these experiments with wortmannin, as well as other experimental evidence, shows that PI3K activity in cells of hematopoietic lineage, particularly neutrophils, monocytes, and other types of leukocytes, is involved in many of the non-memory immune response associated with acute and chronic inflammation.

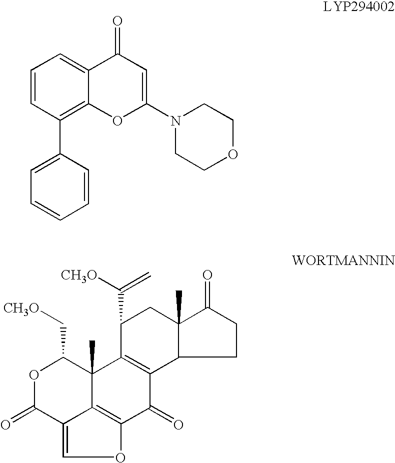

Based on studies using wortmannin, there is evidence that PI3-kinase function is also required for some aspects of leukocyte signaling through G-protein coupled receptors (Thelen et al., 1994, above). Moreover, it has been shown that wortmannin and LY294002 block neutrophil migration and superoxide release. Cyclooxygenase inhibiting benzofuran derivatives are disclosed by John M. Janusz et al., in J. Med. Chem. 1998; Vol. 41, No. 18.

It is now well understood that deregulation of onocogenes and tumour-suppressor genes contributes to the formation of malignant tumours, for example by way of increase cell proliferation or increased cell survival. It is also now known that signaling pathways mediated by the PI3k family have a central role in a number of cell processes including proliferation and survival, and deregulation of these pathways is a causative factor a wide spectrum of human cancers and other diseases (Katso et al., Annual Rev. Cell Dev. Biol. 2001, 17: 615-617 and Foster et al., J. Cell Science, 2003, 116: 3037-3040).

Class I PI3K is a heterodimer consisting of a p110 catalytic subunit and a regulatory subunit, and the family is further divided into class Ia and Class Ib enzymes on the basis of regulatory partners and mechanism of regulation. Class Ia enzymes consist of three distinct catalytic subunits (p110α, p110β, and p110δ) that dimerise with five distinct regulatory subunits (p85α, p55α, p50α, p85β, and p55γ), with all catalytic subunits being able to interact with all regulatory subunits to form a variety of heterodimers. Class Ia PI3K are generally activated in response to growth factor-stimulation of receptor tyrosine kinases, via interaction of the regulatory subunit SH2 domains with specific phosphor-tyrosine residues of the activated receptor or adaptor proteins such as IRS-1. Both p110α and p110β are constitutively expressed in all cell types, whereas p110δ expression is more restricted to leukocyte populations and some epithelial cells. In contrast, the single Class Ib enzyme consists of a p110γ catalytic subunit that interacts with a p101 regulatory subunit. Furthermore, the Class Ib enzyme is activated in response to G-protein coupled receptor (GPCR) systems and its expression appears to be limited to leucoccytes.

There is now considerable evidence indicating that Class Ia PI3K enzymes contribute to tumourigenesis in a wide variety of human cancers, either directly or indirectly (Vivanco and Sawyers, Nature Reviews Cancer, 2002, 2, 489-501). For example, the p110α subunit is amplified in some tumours such as those of the ovary (Shayesteh, et al., Nature Genetics, 1999, 21: 99-102) and cervix (Ma et al., Oncogene, 2000, 19: 2739-2744). More recently, activating mutations within p110α have been associated with various other tumors such as those of the colorectal region and of the breast and lung (Samuels, et al., Science, 2004, 304, 554). Tumor-related mutations in p85α have also been identified in cancers such as those of the ovary and colon (Philp et al., Cancer Research, 2001, 61, 7426-7429). In addition to direct effects, it is believed that activation of Class Ia PI3K contributes to tumourigenic events that occur upstream in signaling pathways, for example by way of ligan-dependent or ligand-independent activation of receptor tyrosine kinases, GPCR systems or integrins (Vara et al., Cancer Treatment Reviews, 2004, 30, 193-204). Examples of such upstream signaling pathways include over-expression of the receptor tyrosine kinase Erb2 in a variety of tumors leading to activation of PI3K-mediated pathways (Harari et al., Oncogene, 2000, 19, 6102-6114) and over-expression of the oncogene Ras (Kauffmann-Zeh et al., Nature, 1997, 385, 544-548). In addition, Class Ia PI3Ks may contribute indirectly to tumourigenesis caused by various downstream signaling events. For example, loss of the effect of the PTEN tumor-suppressor phosphatase that catalyses conversion of PI(3,4,5)P3 back to PI(4,5)P2 is associated with a very broad range of tumors via deregulation of PI3K-mediated production of PI(3,4,5)P3 (Simpson and Parsons, Exp. Cell Res., 2001, 264, 29-41). Furthermore, augmentation of the effects of other PI3K-mediated signaling events is believed to contribute to a variety of cancers, for example by activation of AKT (Nicholson and Andeson, Cellular Signaling, 2002, 14, 381-395).

In addition to a role in mediating proliferative and survival signaling in tumor cells, there is also good evidence that class Ia PI3K enzymes will also contribute to tumourigenesis via its function in tumor-associated stromal cells. For examples, PI3K signaling is known to play an important role in mediating angiogenic events in endothelial cells in response to pro-angiogenic factors such as VEGF (abid et al., Arterioscler. Thromb. Vasc. Biol., 2004, 24, 294-300). As Class I PI3K enzymes are also involved in motility and migration (Sawyer, Expert Opinion investing. Drugs, 2004, 13, 1-19), PI3K inhibitors should provide therapeutic benefit via inhibition of tumor cell invasion and metastasis.

### DESCRIPTION OF THE RELATED ART

U.S. application Ser. No. 60/677284, filed May 3, 2005, describes a group of thiazolidinone compounds which are indicated as having hYAK3 inhibitory activity and which are indicated as being useful in the treatment of deficiencies in hematopoietic cells, in particular in the treatment of deficiencies in erythroid cells.

U.S. application Ser. No. 60/677284 does not disclose the use of any of the compounds described therein as inhibitors or inhibitors of PI3 kinases.

### SUMMARY OF THE INVENTION

This invention relates to a method of inhibiting one or more PI3 kinases selected from: PI3Kα, PI3Kδ, P/I3Kβ and PI3Kγ, in a mammal in need thereof, which method comprises administrating to such mammal a therapeutically effective amount of a compound of Formula (I):

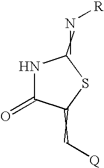

in which

R is selected form: aryl and substituted aryl; and
Q is

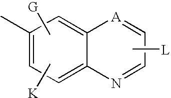

wherein

A is selected from CR⁵⁰ and N,
where R⁵⁰, G, K and L are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 4 heteroatoms, substituted cycloalkyl containing from 1 to 4 heteroatoms, —C(O)OR¹⁰, —C(O)NR¹¹R¹² and cyano,
where, R¹⁰ is selected form hydrogen, C₁-C₄alkyl, aryl and trifluoromethyl, and R¹¹ and R¹² are independently selected form hydrogen, C₁-C₄alkyl, aryl and trifluoromethyl,
provided that at least one of G, K, L and R⁵⁰, when R⁵⁰ is present, is not hydrogen;
   and/or pharmaceutically acceptable salts, hydrates, solvates and pro-drugs thereof.

This invention also relates to a method of treating cancer, which comprises administering to a subject in need thereof an effective amount of a compound of Formula (I).

This invention also relates to a method of treating one or more disease states selected from: autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection and lung injuries, which comprises administering to a subject in need thereof an effective amount of a compound of Formula (I).

Included in the present invention are methods of co-administering the present PI3 kinase inhibiting compounds with further active ingredients.

### DETAILED DESCRIPTION OF THE INVENTION

Present compounds of Formula (I) inhibit PI3 kinase. Suitably, the compounds of Formula (I) inhibit one or more PI3 kinases selected from: PI3Kα, PI3Kδ, PI3Kβ and PI3Kγ.

Included among the compounds of Formula (I) that are active as inhibitors of PI3 kinase activity are those having Formula (II): Included among the presently invented compounds of Formula (I) are those having Formula (II):

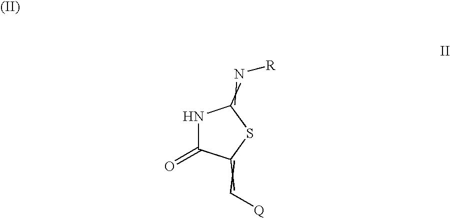

in which

R is selected form: C₁-C₁₂aryl and substituted C₁-C₁₂aryl; and
Q is

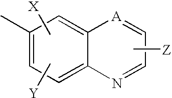

wherein

A is selected from CR⁵¹ and N,
where R⁵¹, X, Y and Z are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 3 heteroatoms, substituted cycloalkyl containing from 1 to 3 heteroatoms, —C(O)OR¹⁰, —C(O)NR¹¹R¹² and cyano,
where R¹⁰ is selected form hydrogen, C₁-C₄alkyl, aryl and trifluoromethyl, and R¹ 1 and R¹² are independently selected form hydrogen, C₁-C₄alkyl, aryl and trifluoromethyl,
provided that at least one of X, Y, Z and R⁵¹, when R⁵¹ is present, is not hydrogen;
   and/or pharmaceutically acceptable salts, hydrates, solvates and pro-drugs thereof.

Included among the presently invented compounds of Formulas (I) and (II) are those in which:
R is

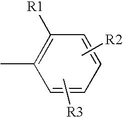

in which R¹ is hydrogen, halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, —OCF₃, or —CO₂C₁₋₆alkyl; and
R² and R³ are independently hydrogen, halogen, —C₁₋₆ alkyl, substituted —C₁₋₆alkyl, C₁₋₁₂aryl, cycloalkyl, cycloalkyl containing from 1 to 4 heteroatoms, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, —CO₂C₁₋₆alkyl, —NH₂, alkylamino, dialkylamino, —OCH₂(C═O)OH, —OCH₂CH₂OCH₃, —SO₂NH₂,
—S(O)₂NR⁴⁰R³⁰, where R³⁰ is selected from alkyl, cycloalkyl, substituted cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl,
—NR⁴¹C(O)R³¹, where R³¹ is selected from aryl, —Oalkyl, —Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl, and R⁴¹ is selected from hydrogen and C₁-C₆alkyl,
—NR⁴⁴S(O)₂R³⁴, where R³⁴ is selected from hydrogen, alkyl, cycloalkyl, substituted cycloalkyl, cycloalkyl containing 1 to 4 heteroatoms, substituted cycloalkyl containing 1 to 4 heteroatoms and aryl, and R⁴⁴ is selected from hydrogen and C₁-C₆alkyl,
—CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl,
—NH(C═NH)CH₃; and
Q is

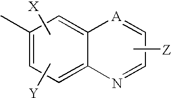

wherein

A is selected from CR⁵¹ and N,
where R⁵¹, X, Y and Z are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 3 heteroatoms, substituted cycloalkyl containing from 1 to 3 heteroatoms, —C(O)OR¹⁰, —C(O)NR¹¹R¹² and cyano,
where R¹⁰ is selected form hydrogen, C₁-C₄alkyl, aryl and trifluoromethyl, and R¹¹ and R¹² are independently selected form hydrogen, C₁-C₄alkyl, aryl and trifluoromethyl,
provided that at least one of X, Y, Z and R⁵¹, when R⁵¹ is present, is not hydrogen;
   and/or pharmaceutically acceptable salts, hydrates, solvates and pro-drugs thereof.

Included among the presently invented compounds of Formulas (I) and (II) are those in which:

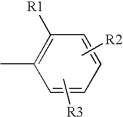

in which R1 is halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, or —CO₂C₁₋₆alkyl;
and R2 and R3 are independently hydrogen, halogen, —C₁₋₆ alkyl, substituted —C₁₋₆ alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, —S(O)₂NR⁴⁰R³⁰, where R³⁰ is selected from alkyl, cycloalkyl, substituted cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl,
—NR⁴¹C(O)R³¹, where R³¹ is selected from aryl, —Oalkyl, —Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl, and R⁴¹ is selected from hydrogen and C₁-C₆alkyl,
—NR⁴⁴S(O)₂R³⁴, where R³⁴ is selected from hydrogen, alkyl, cycloalkyl, substituted cycloalkyl, cycloalkyl containing 1 to 4 heteroatoms, substituted cycloalkyl containing 1 to 4 heteroatoms and aryl, and R⁴⁴ is selected from hydrogen and C₁-C₆alkyl,
—CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl,
—CO₂C₁₋₆alkyl, —NH₂, alkylamino, dialkylamino or —NH(C═NH)CH₃;
and
Q is

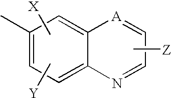

wherein

A is selected from CR⁵¹ and N,
where R⁵¹, X, Y and Z are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 3 heteroatoms, substituted cycloalkyl containing from 1 to 3 heteroatoms, —C(O)OR¹⁰, —C(O)NR ¹¹R¹² and cyano,
where R¹⁰ is selected form hydrogen, C₁-C₄alkyl, aryl and trifluoromethyl, and R¹¹ and R¹² are independently selected form hydrogen, C₁-C₄alkyl, aryl and trifluoromethyl,
provided that at least one of X, Y, Z and R⁵¹, when R⁵¹ is present, is not hydrogen;
   and/or pharmaceutically acceptable salts, hydrates, solvates and pro-drugs thereof.

Included among the presently invented compounds of Formulas (I) and (II) are those in which:
R is

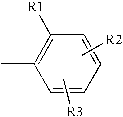

in which R1 is halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, or —CO₂C₁₋₆alkyl; and R2 and R3 are independently hydrogen, halogen, —C₁₋₆ alkyl, substituted —C₁₋₆ alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, —S(O)₂NR⁴⁰R³⁰, where R³⁰ is selected from alkyl, cycloalkyl, substituted cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl,
—NR⁴¹C(O)R³¹, where R³¹ is selected from aryl, —Oalkyl, —Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl, and R⁴¹ is selected from hydrogen and C₁- C₆alkyl,
—NR⁴⁴S(O)₂R³⁴, where R³⁴ is selected from hydrogen, alkyl, cycloalkyl, substituted cycloalkyl, cycloalkyl containing 1 to 4 heteroatoms, substituted cycloalkyl containing 1 to 4 heteroatoms and aryl, and R⁴⁴ selected from hydrogen and C₁-C₆alkyl,
—CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl,
—CO₂C₁₋₆alkyl, —NH₂, alkylamino or —NH(C═NH)CH₃;
and
Q is

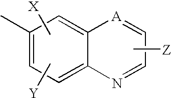

wherein

A is selected from CR⁵¹ and N,
where R⁵¹, X, Y and Z are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 3 heteroatoms, substituted cycloalkyl containing from 1 to 3 heteroatoms, —C(O)OR¹⁰, —C(O)NR¹¹R¹² and cyano,
where R¹⁰ is selected form hydrogen, C₁-C₄alkyl, aryl and trifluoromethyl, and R¹¹ and R¹² are independently selected form hydrogen, C₁-C₄alkyl, aryl and trifluoromethyl,
provided that at least one of X, Y, Z and R⁵¹, when R⁵¹ is present, is not hydrogen;
   and/or pharmaceutically acceptable salts, hydrates, solvates and pro-drugs thereof.

Included among the presently invented compounds of Formulas (I) and (II) are those in which:
R is

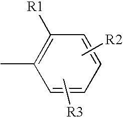

in which R1 is halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —S(═O)—C₁₋₆alkyl, —OH, —CF₃, —CN, —CO₂H, or —CO₂C₁₋₆alkyl; and R2 and R3 are independently hydrogen, halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —OH, —CF₃, —CN, —CO₂H, —S(O)₂NR⁴⁰R³⁰, where R³⁰ is selected from alkyl, cycloalkyl, substituted cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl,
—NR⁴¹C(O)R³¹, where R³¹ is selected from aryl, —Oalkyl, —Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl, and R⁴¹ is selected from hydrogen and C₁-C₆alkyl,
—NR³⁴S(O)₂R³⁴, where R³⁴ is selected from hydrogen, alkyl, cycloalkyl, substituted cycloalkyl, cycloalkyl containing 1 to 4 heteroatoms, substituted cycloalkyl containing 1 to 4 heteroatoms and aryl, and R⁴⁴ is selected from hydrogen and C₁-C₆alkyl, —CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl,
—CO₂C₁₋₆alkyl, —NH₂, alkylamino, or —NH(C═NH)CH₃;
and

### Q is

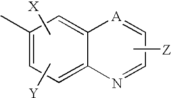

wherein

A is selected from CR⁵¹ and N,
where R⁵¹, X, Y and Z are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 3 heteroatoms, substituted cycloalkyl containing from 1 to 3 heteroatoms, —C(O)OR¹⁰, —C(O)NR¹¹R¹² and cyano,
   where R¹⁰ is selected form hydrogen, C₁-C₄alkyl, aryl and trifluoromethyl, and R¹¹ and R¹² are independently selected form hydrogen, C₁-C₄alkyl, aryl and trifluoromethyl,
   provided that at least one of X, Y, Z and R⁵¹, when R⁵¹ is present, is not hydrogen;
      and/or pharmaceutically acceptable salts, hydrates, solvates and pro-drugs thereof.

Included among the novel compounds useful in the present invention are:
(5Z)-2-[(2,6-Dichlorophenyl)amino]-5-{[3-(4-morpholinyl)-6-quinoxalinyl]methylidene}-1,3-thiazol-4(5H)-one;
7-{(Z)-[2-[(2,6-Dichlorophenyl)amino]-4-oxo-1,3-thiazol-5(4H)-ylidene]methyl}-2(1H)-quinoxalinone;
(5Z)-2-[(2,6-Dichlorophenyl)amino]-5-{[2-(methylamino)-6-quinolinyl]methylidene}-1,3-thiazol-4(5H)-one;
(5Z)-2-[(2,6-Dichlorophenyl)amino]-5-{[4-(dimethylamino)-6-quinolinyl]methylidene}-1,3-thiazol-4(5H)-one; and
(5Z)-2-[(2,6-Dichlorophenyl)amino]-5-{[4-(4-methyl-1-piperazinyl)-6-quinolinyl]methylidene}-1,3-thiazol-4(5H)-one.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Compounds of Formula (I) are included in the pharmaceutical compositions of the invention.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant phenyl, naphthalene, 3,4-methylenedioxyphenyl, pyridine, biphenyl, quinoline, pyrimidine, quinazoline, thiophene, thiazole, furan, pyrrole, pyrazole, imidazole, indole, indene, pyrazine, 1,3-dihydro-2H-benzimidazol, benzimidazol, benzothiohpene, tetrahydrobenzothiohpene and tetrazole.

The term “substituted” as used herein, unless otherwise defined, is meant that the subject chemical moiety has one or more substituents selected from the group consisting of: aryl,
aryl substituted with one or more substituents selected from alkyl, hydroxy, alkoxy, oxo, C₁-C₁₂aryl optionally substituted with one or more substituents selected from hydroxy, alkoxy oxo, cyano, amino, alkylamino, dialkylamino, alkyl and alkoxy, trifluoromethyl, —SO₂NR²¹R²², N-acylamino, —CO₂R²⁰, and halogen, cycloalkyl substituted with one or more substituents selected from alkyl, hydroxy, alkoxy, trifluoromethyl, —SO₂NR²¹R²², amino, —CO₂R²⁰, N-acylamino and halogen,
cycloalkyl containing from 1 to 4 heteroatoms substituted with one or more substituents selected from alkyl, hydroxy, alkoxy, —SO₂NR²¹R²², amino, —CO₂R²⁰, trifluoromethyl, N-acylamino and halogen,
alkoxy substituted with one or more substituents selected form alkyl, —CO₂H, hydroxyl, C₁-C₁₂aryl, alkoxy, amino and halogen,
cycloalkyl, cycloalkyl containing from 1 to 4 heteroatoms, C₁-C₄alkylcycloalkyl containing from 1 to 3 heteroatomsC₁-C₄alkyl, —C(O)NHS(O)₂R²⁰, —(CH₂)_{g}NR²³S(O)₂R²⁰, hydroxyalkyl, alkoxy, —(CH₂)_{g}NR²¹R²², —C(O)NR²¹R²², —S(O)₂NR²¹R²², —(CH₂)_{g}N(R²⁰)C(O)ₙR²⁰, —(CH₂)_{g}N═C(H)R²⁰, —C(O)R²⁰, acyloxy, —SC₁-C₆alkyl, alkyl, —OCF₃, amino, hydroxy, alkylamino, acetamide, aminoalkyl, aminoalkoxy, alkylaminoalkoxy, dialkylaminoalkoxy, alkoxyalkylamide, alkoxyC₁-C₁₂aryl, C₁-C₁₂aryl, C₁-C₁₂arylalkyl, dialkylamino, N-acylamino, aminoalkylN-acylamino, —(CH₂)_{g}C(O)OR²⁰, —(CH₂)_{g}S(O)ₙR²³, nitro, cyano, oxo, halogen, trifluoromethyloxy and trifluoromethyl;
where g is 0 to 6, n is 0 to 2, R²³ is hydrogen or alkyl, each R²⁰ is independently selected form hydrogen, alkyl, C₁- C₆alkyloxyC₁-C₆alkyl, C₁-C₄alkylC(O)OC₁-C₄alkyl, amino, alkylamino, dialkylamino, aminoC₁-C₆alkyl, alkylaminoC₁-C₆alkyl, dialkylaminoC₁-C₆alkyl, —C(O)OH, alkoxy, aryloxy, arylamino, diarylamino, arylalkylamino, aryl, aryl substituted with one or more substituents selected from oxo, hydroxyl and alkyl, arylC₁-C₄alkyl optionally substituted with one or more substituents selected from oxo, hydroxy, halogen, alkoxy and alkyl, —CH₂C(O)cycloalkyl containing from 1 to 4 heteroatoms, cycloalkylC₁-C₄alkyl, C₁-C₄alkyl substituted with cycloalkyl containing from 1 to 4 heteroatoms, cycloalkyl, cycloalkyl substituted with one or more substituents selected from oxo, hydroxyl and alkyl, cycloalkyl containing from 1 to 4 heteroatoms, cycloalkyl containing from 1 to 4 heteroatoms substituted with one or more substituents selected from oxo, hydroxyl and alkyl, and trifluoromethyl, and R²¹ and R²² are independently selected form hydrogen, alkyl, C₁-C₆alkyl substituted with one or more substituents selected from hydroxy, amino, ═NH, and ≡N, —S(O)₂aryl, —S(O)₂alkyl, C₁-C₁₂aryl, cycloalkyl containing from 1 to 4 heteroatoms, cycloalkyl containing from 1 to 4 heteroatoms substituted with one or more substituents selected from oxo, hydroxy, and alkyl, cycloalkyl, cycloalkyl substituted with one or more substituents selected from oxo, hydroxy, and alkyl, arylC₁-C₆alkyl optionally substituted with one or more substituents selected from oxo, hydroxy, and alkyl, cycloalkyl containing from 1 to 4 heteroatoms optionally substituted with one or more substituents selected from oxo, hydroxyl and alkyl, C₁-C₆alkoxy, C₁-C₄alkyloxyC₁-C₄alkyl, aryl and trifluoromethyl.

By the term “naphthyridin-6-yl” as used herein, is meant 1,5-naphthyridin-6-yl, 1,7-naphthyridin-6-yl, and 1,8-naphthyridin-6-yl.

By the term “alkoxy” as used herein is meant —Oalkyl where alkyl is as described herein including —OCH₃ and —OC(CH₃)₂CH₃.

The term “cycloalkyl” as used herein unless otherwise defined, is meant a nonaromatic, unsaturated or saturated, cyclic or polycyclic C₃-C₁₂.

Examples of cycloalkyl and substituted cycloalkyl substituents as used herein include: cyclohexyl, aminocyclohexyl, cyclobutyl, aminocyclobutyl, 4-hydroxy-cyclohexyl, 2-ethylcyclohexyl, propyl4-methoxycyclohexyl, 4-methoxycyclohexyl, 4-carboxycyclohexyl, cyclopropyl, aminocyclopentyl, and cyclopentyl.

The term “cycloalkyl containing from 1 to 4 heteroatoms” and the term “cycloalkyl containing from 1 to 3 heteroatoms” as used herein unless otherwise defined, is meant a nonaromatic, unsaturated or saturated, cyclic or polycyclic ring containing from 1 to 12 carbons and containing from one to four heteroatoms or from one to three heteroatoms (respectively), provided that when the number of carbon atoms is 1 the aromatic ring contains at least four heteroatoms (applicable only where “cycloalkyl containing from 1 to 4 heteroatoms” is indicated), when the number of carbon atoms is 2 the aromatic ring contains at least three heteroatoms, when the number of carbon atoms is 3 the nonaromatic ring contains at least two heteroatoms and when the number of carbon atoms is 4 the nonaromatic ring contains at least one heteroatom.

Examples of cycloalkyl containing from 1 to 4 heteroatoms, cycloalkyl containing from 1 to 3 heteroatoms, substituted cycloalkyl containing from 1 to 4 heteroatoms and substituted cycloalkyl containing from 1 to 3 heteroatoms as used herein include: piperidine, piperazine, pyrrolidine, 3-methylaminopyrrolidine, piperazine, tetrazole, hexahydrodiazepine and morpholine.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include: —N(H)C(O)CH₃, —N(H)C(O)CH(CH₃)₂ and —N(H)C(O)(CH₂)₃CH₃.

By the term “aryloxy” as used herein is meant —Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifuloromethyl, acyloxy, amino, N-acylamino, hydroxy, —(CH₂)_{g}C(O)OR²⁵, —S(O)ₙR²⁵, nitro, cyano, halogen and protected —OH, where g is 0-6, R²⁵ is hydrogen or alkyl, and n is 0-2. Examples of aryloxy substituents as used herein include: phenoxy, 4-fluorophenyloxy and biphenyloxy.

By the term “heteroatom” as used herein is meant oxygen, nitrogen or sulfur.

By the term “halogen” as used herein is meant a substituent selected from bromide, iodide, chloride and fluoride.

By the term “alkyl” and derivatives thereof and in all carbon chains as used herein, including alkyl chains defined by the term “—(CH₂)ₙ”, “—(CH₂)ₘ” and the like, is meant a linear or branched, saturated or unsaturated hydrocarbon chain, and unless otherwise defined, the carbon chain will contain from 1 to 12 carbon atoms.

Examples of alkyl and substituted alkyl substituents as used herein include:
—CH₃, —CH₂—CH₃, —CH₂—CH₂—CH₃, —CH(CH₃)₂, —CH₂—CH₂—C(CH₃)₃, —CH₂—CF₃, —C≡—C—C(CH₃)₃, —C≡C—CH₂—OH, cyclopropylmethyl, —CH₂—C(CH₃)₂—CH₂—NH₂, C≡C—C₆H₅, —C≡C—C(CH₃)₂—OH, —CH₂—CH(OH)—CH(OH)—CH(OH)—CH(OH)—CH₂—OH, piperidinylmethyl, methoxyphenylethyl, —C(CH₃)₃, —(CH₂)₃—CH₃, —CH₂—CH(CH₃)₂, —CH(CH₃)—CH₂—CH₃, —CH═CH₂, and —C≡C—CH₃.

By the term “treating” and derivatives thereof as used herein, is meant prophylatic and therapeutic therapy.

As used herein, the term “optionally” means that the subsequently described event(s) may or may not occur, and includes both event(s), which occur, and events that do not occur.

As used herein, the crisscrossed double bond indicated by the symbol [symbol] denotes Z and/or E stereochemistry around the double bond. In other words a compound of formula I or II can be either in the Z or E stereochemistry around this double bond, or a compound of formula I or II can also be in a mixture of Z and E stereochemistry around the double bond. However, in formulas I and II, the preferred compounds have Z stereochemistry around the double bond to which radical Q is attached.

The compounds of Formulas I and II naturally may exist in one tautomeric form or in a mixture of tautomeric forms. For example, for sake simplicity, compounds of formula I and II are expressed in one tautomeric form, usually as an exo form, i.e.

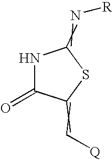

### Exo form

However, a person of ordinary skill can readily appreciate, the compounds of formulas I and II can also exist in endo forms.

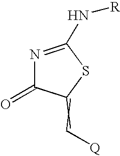

### Endo form

The present invention contemplates all possible tautomeric forms.

Certain compounds described herein may contain one or more chiral atoms, or may otherwise be capable of existing as two enantiomers, or two or more diastereoisomers. Accordingly, the compounds of this invention include mixtures of enantiomers/diastereoisomers as well as purified enantiomers/diastereoisomers or enantiomerically/diastereoisomerically enriched mixtures. Also included within the scope of the invention are the individual isomers of the compounds represented by formula I or II above as well as any wholly or partially equilibrated mixtures thereof. The present invention also covers the individual isomers of the compounds represented by the formulas above as mixtures with isomers thereof in which one or more chiral centers are inverted. Further, an example of a possible tautomer is an oxo substituent in place of a hydroxy substituent. Also, as stated above, it is understood that all tautomers and mixtures of tautomers are included within the scope of the compounds of Formula I or II.

Compounds of Formula (I) are included in the pharmaceutical compositions of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or prodrug formulations.

The novel compounds of Formulas I and II are prepared as shown in Schemes I and II below, or by analogous methods, wherein the ‘Q’ and ‘R’ substituents are as defined in Formulas I and II respectively and provided that the ‘Q’ and ‘R’ substituents do not include any such substituents that render inoperative the processes of Schemes I to II. All of the starting materials are commercially available or are readily made from commercially available starting materials by those of skill in the art.

### General Schemes

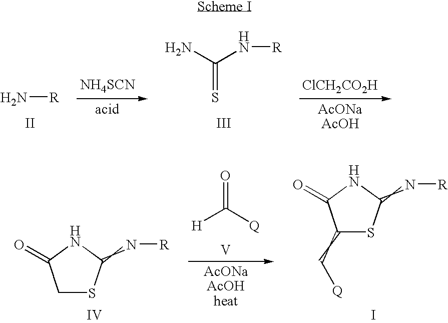

Briefly in Scheme 1, a mixture of aniline derivative of formula II (1 equivalent) and NH4SCN (about 1.3 equivalent) in an acid (typically 4N-HCl) is heated to reflux at about 110° C. for 6 hours. After cooling, the mixture is treated with H₂O, which process usually forms a solid, followed by desiccation in vacuo to give a compound of formula III.

A mixture of formula III compound, CICH₂CO₂H (1 equivalent), and AcONa (1 equivalent) in AcOH is heated to reflux at around 110° C. for about 4 h. The mixture is poured onto water thereby a solid is typically formed, which is isolated by filtration. The solid is washed with a solvent such as MeOH to afford a compound of formula IV.

A mixture of formula IV compound, an aldehyde of formula V (1 equivalent), AcONa (3 equivalent) in AcOH is heated to reflux at about 110° C. for about 10 to 48 hours. After cooling, a small portion of water is added until the solid forms. The solid is filtered and washed with a solvent such as MeOH, followed by desiccation in vacuo to afford a target product of Formula I.

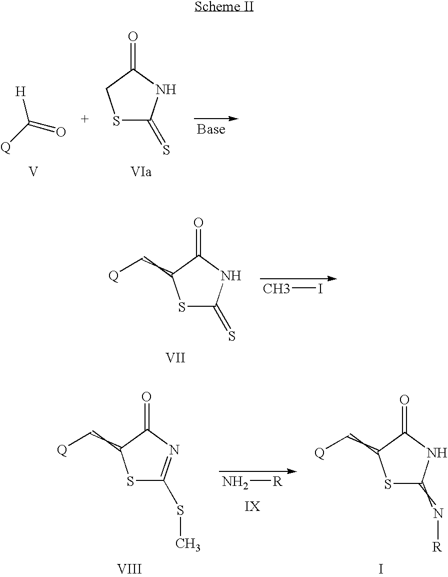

Briefly in Scheme 2, a mixture of an aldehyde of formula V (1 equivalent ), Rhodanine (1 equivalent), sodium acetate (about 3 equivalents), and acetic acid is heated at around 110° C. for about 48 h. The reaction mixture is cooled to room temperature to afford a product of formula VII.

Then, to a room temperature suspension of VII (1 equivalent) in a suitable solvent such as ethanol is added Hunig's base (about 2 equivalents) followed by iodomethane (about 5 equivalents). The resultant suspension is stirred at room temperature for 3.5 h to yield a compound of VIII.

To a mixture of VIII (1 equivalent) and MS4A powder is added an amine of formula IX (1-2 equivalent) and ethanol (dehydrated). The mixture is heated by microwave (SmithSynthesizer-Personal Chemistry) at about 110° C. for about 1200 seconds. The desired product of Formula I is obtained in about 20-90% yield after purification.

In Schemes 1 and 2, the meaning of R and Q are as defined in Formula I.

In other embodiments, additional compounds of the invention can also be synthesized whereby a compound of Formula I is first made by a process of Scheme 1 or 2 (or a variant thereof), and Q and R radicals in compounds of Formula I thus made are further converted by routine organic reaction techniques into different Q and R groups.

It has now been found that compounds of the present invention are inhibitors of the Phosphatoinositides 3-kinases (PI3Ks). When the phosphatoinositides 3-kinase (PI3K) enzyme is inhibited by a compound of the present invention, PI3K is unable to exert its enzymatic, biological and/or pharmacological effects. The compounds of the present invention are therefore useful in the treatment of autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries.

The compounds of Formula (I) are useful as medicaments in particular for the treatment of autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries. According to one embodiment of the present invention, the compounds of Formula (I) are inhibitors of one or more phosphatoinositides 3-kinases (PI3Ks), suitably, Phosphatoinositides 3-kinase γ (PI3Kγ), Phosphatoinositides 3-kinase γ (PI3Kα), Phosphatoinositides 3-kinase γ (PI3Kβ), and/or Phosphatoinositides 3-kinase γ (PI3Kδ).

Compounds according to Formula (I) are suitable for the modulation, notably the inhibition of the activity of phosphatoinositides 3-kinases (PI3K), suitably phosphatoinositides 3-kinase (PI3Kγ). Therefore the compounds of the present invention are also useful for the treatment of disorders which are mediated by PI3Ks. Said treatment involves the modulation—notably the inhibition or the down regulation—of the phosphatoinositides 3-kinases.

Suitably, the compounds of the present invention are used for the preparation of a medicament for the treatment of a disorder selected from multiple sclerosis, psoriasis, rheumatoid arthritis, systemic lupus erythematosis, inflammatory bowel disease, lung inflammation, thrombosis or brain infection/inflammation, such as meningitis or encephalitis, Alzheimer's disease, Huntington's disease, CNS trauma, stroke or ischemic conditions, cardiovascular diseases such as athero-sclerosis, heart hypertrophy, cardiac myocyte dysfunction, elevated blood pressure or vasoconstriction.

Suitably, the compounds of Formula (I) are useful for the treatment of autoimmune diseases or inflammatory diseases such as multiple sclerosis, psoriasis, rheumatoid arthritis, systemic lupus erythematosis, inflammatory bowel disease, lung inflammation, thrombosis or brain infection/inflammation such as meningitis or encephalitis.

Suitably, the compounds of Formula (I) are useful for the treatment of neurodegenerative diseases including multiple sclerosis, Alzheimer's disease, Huntington's disease, CNS trauma, stroke or ischemic conditions.

Suitably, the compounds of Formula (I) are useful for the treatment of cardiovascular diseases such as atherosclerosis, heart hypertrophy, cardiac myocyte dysfunction, elevated blood pressure or vasoconstriction.

Suitably, the compounds of Formula (I) are useful for the treatment of chronic obstructive pulmonary disease, anaphylactic shock fibrosis, psoriasis, allergic diseases, asthma, stroke, ischemic conditions, ischemia-reperfusion, platelets aggregation/activation, skeletal muscle atrophy/hypertrophy, leukocyte recruitment in cancer tissue, angiogenesis, invasion metastasis, in particular melanoma, Karposi's sarcoma, acute and chronic bacterial and virual infections, sepsis, transplantation rejection, graft rejection, glomerulo sclerosis, glomerulo nephritis, progressive renal fibrosis, endothelial and epithelial injuries in the lung, and lung airway inflammation.

Because the pharmaceutically active compounds of the present invention are active as PI3 kinase inhibitors, particularly the compounds that modulate/inhibit PI3Kα, either selectively or in conjunction with one or more of PI3Kδ, PI3Kβ, and/or PI3Kγ, they exhibit therapeutic utility in treating cancer.

Suitably, the present invention relates to a method for treating or lessening the severity of a cancer selected from brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, pancreatic, breast, prostate and leukemia.

When a compound of Formula (I) is administered for the treatment of cancer, the term “co-administering” and derivatives thereof as used herein is meant either simultaneous administration or any manner of separate sequential administration of a PI3 kinase inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of cancer in the present invention. Examples of such agents can be found in Cancer Principles and Practice f Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the present PI3 kinase inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7,β,10β, 13α-hexa-hydroxytax-11-en-9-one 4,10-diacetate 2-benzoate 13-ester with (2R,3S)-N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273,1989) and for the treatment of breast cancer (Holmes et al., J. Nat. Cancer Inst., 83:1797,1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p.16-23, 1995).

Docetaxel, (2R,3S)-N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*, R*)—2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)—O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′, 2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H, 12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H, 12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula A following, currently under development, including the racemic mixture (R,S) form as well as the R and S enantiomers:

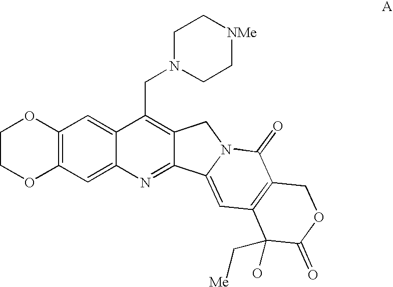

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children ; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor —I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 February 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60.1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin ® erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the cancer treatment method of the claimed invention includes the co-administration a compound of formula I and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

Because the pharmaceutically active compounds of the present invention are active as PI3 kinase inhibitors, particularly the compounds that inhibit PI3Kγ, either selectively or in conjunction with one or more of PI3Kδ, PI3Kβ, and/or PI3Kα, they exhibit therapeutic utility in treating a disease state selected from: autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection and lung injuries.

When a compound of Formula (I) is administered for the treatment of a disease state selected from: autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection or lung injuries, the term “co-administering” and derivatives thereof as used herein is meant either simultaneous administration or any manner of separate sequential administration of a PI3 kinase inhibiting compound, as described herein, and a further active ingredient or ingredients, known to be useful in the treatment of autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection and/or lung injuries.

### Biological Assays

The compounds of the present invention are tested to determine their inhibitory activity at PI3Kα, PI3Kδ, PI3Kβ and PI3Kγ according to the following.

For all PI3K isoforms:
1. Cloning, expression, purification, and characterization of the human Class Ia phosphoinositide 3-kinase isoforms: Meier, T. I.; Cook, J. A.; Thomas, J. E.;

Radding, J. A.; Horn, C.; Lingaraj, T.; Smith, M. C. Protein Expr. Purif., 2004, 35(2), 218.
2. Competitive fluorescence polarization assays for the detection of phosphoinositide kinase and phosphatase activity: Drees, B. E.; Weipert, A.;

Hudson, H.; Ferguson, C. G.; Chakravarty, L.; Prestwich, G. D. Comb. Chem. High Throughput.Screen., 2003, 6(4), 321.

### For PI3Kγ: WO 2005/011686 A1

The pharmaceutically active compound within the scope of this invention are useful as PI3 Kinase inhibitors in mammals, particularly humans, in need thereof.

The present invention therefore provides a method of treating diseases associated with PI3 kinase modulation/inhibition, particularly: autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries and other conditions requiring PI3 kinase modulation/inhibition, which comprises administering an effective compound of Formula (I) or a pharmaceutically acceptable salt, hydrate, solvate or pro-drug thereof. The compounds of Formula (I) also provide for a method of treating the above indicated disease states because of their ability to act as PI3 inhibitors. The drug may be administered to a patient in need thereof by any conventional route of administration, including, but not limited to, intravenous, intramuscular, oral, subcutaneous, intradermal, and parenteral.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of a PI3K inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages is preferred. Parenteral administration, at high dosages, however, also can be used when safe and convenient for the patient. The above dosages relate to suitable amount of compound expressed as the free acid.

Optimal dosages to be administered may be readily determined by those skilled in the art, and will vary with the particular PI3 kinase inhibitor in use, the strength of the preparation, the mode of administration, and the advancement of the disease condition. Additional factors depending on the particular patient being treated will result in a need to adjust dosages, including patient age, weight, diet, and time of administration.

The method of this invention of inducing PI3 kinase inhibitory activity in mammals, including humans, comprises administering to a subject in need of such activity an effective PI3 kinase modulating/inhibiting amount of a pharmaceutically active compound of the present invention.

The invention also provides for the use of a compound of Formula (I) in the manufacture of a medicament for use as a PI3 kinase inhibitor.

The invention also provides for the use of a compound of Formula (I) in the manufacture of a medicament for use in therapy.

The invention also provides for the use of a compound of Formula (I) in the manufacture of a medicament for use in treating autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries.

The invention also provides for a pharmaceutical composition for use as a PI3 inhibitor which comprises a compound of Formula (I) and a pharmaceutically acceptable carrier.

The invention also provides for a pharmaceutical composition for use in the treatment of autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries, which comprises a compound of Formula (I) and a pharmaceutically acceptable carrier.

No unacceptable toxicological effects are expected when compounds of the invention are administered in accordance with the present invention.

In addition, the pharmaceutically active compounds of the present invention can be co-administered with further active ingredients, including compounds known to have utility when used in combination with a PI3 kinase inhibitor.

Without further elaboration, it is believed that one skilled in the art can, using the preceding description, utilize the present invention to its fullest extent. The following examples are, therefore, to be construed as merely illustrative and not a limitation of the scope of the present invention in any way.

For ease of illustration, the regiochemistry around the double bonds in the chemical formulas in the Examples are drawn as fixed for ease of representation; however, a skilled in the art will readily appreciate that the compounds will naturally assume more thermodynamically stable structure around the C═N (the imine) double bond if it exits as exo form. Further compounds can also exit in endo form. As stated before, the invention contemplates both endo and exo forms as well as both regioisomers around the exo imine bond. Further it is intended that both E and Z isomers are encompassed around the C═C double bond.

### Experimental Details

The compounds of Examples 1 to 8 are readily made according to Schemes I and II or by analogous methods.

### EXAMPLE 1

(5Z)-2-[(2,6-Dichlorophenyl)amino]-5-{[3-(4-morpholinyl )-6-quinoxalinyl]methylidene}-1,3-thiazol-4(5H)-one
a) 7-Bromo-2-chloroquinoxaline. A solution of 7-bromo-2(1H)-quinoxalinone (prepared by the method of Linda, P; Marino, G. Ric. Sci., Rend. Sez. A. [2] 1963, 3, 225-228) (0.900 g, 4.00 mmol) was treated with phosphorus oxychloride (5.0 mL). After heating to reflux overnight, the reaction mixture was poured into ice water, extracted thrice with ethyl acetate, dried over magnesium sulfate, filtered, and concentrated in vacuo to give the title compound as an off-white solid (0.905 g, 93%). 1H NMR (400 MHz, DMSO-d₆) δ ppm 9.05 (s, 1 H) 8.34 (d, J=2.0 Hz, 1 H) 8.11 (d, J=9.0 Hz, 1 H) 8.05 (dd, J=8.9, 2.0 Hz, 1 H). MS(ES+) m/e 243/245 [M+H]⁺.
b) 7-Bromo-2-(4-morpholinyl)quinoxaline. A solution of the compound from Example 1a) (5.00 g; 20.5 mmol) and morpholine (1.80 mL; 20.5 mmol) in methanol (4.0 mL) was stirred and heated to 150° C. for 20 min. in a Biotage Initiator microwave synthesizer. Concentration in vacuo and recrystallization from CH₂Cl₂ provided the title compound as a light yellow crystalline solid (5.75 g, 95%). 1H NMR (400 MHz, CHLOROFORM-d) δ ppm 8.45 (s, 1 H) 7.76 (d, J=2.0 Hz, 1 H) 7.64 (d, J=8.6 Hz, 1 H) 7.38 (dd, J=8.7, 2.1 Hz, 1 H) 3.76-3.84 (m, 4 H) 3.67-3.71 (m, 4 H). MS(ES+) m/e 294/296 [M+H]⁺.
c) 7-Ethenyl-2-(4-morpholinyl)quinoxaline. A solution of the compound from Example 1b) (0.362 g; 1.23 mmol), tributyl(vinyl)tin (0.395 mL; 1.35 mmol), and tetrakis(triphenylphosphine)palladium(0) (0.014 g; 0.012 mmol) in dioxane (2.0 mL) was stirred and heated to 150° C. for 20 min. in a Biotage Initiator microwave synthesizer. Concentration in vacuo and purification via flash column chromatography (10-50% ethyl acetate in hexanes) provided the title compound as a yellow solid (0.276 g, 93%). 1H NMR (400 MHz, CHLOROFORM-d) δ ppm 8.51 (s, 1 H) 7.84 (d, J=8.6 Hz, 1 H) 7.66 (d, J=1.5 Hz, 1 H) 7.55 (dd, J=8.5, 1.9 Hz, 1 H) 6.86 (dd, J=17.4, 10.9 Hz, 1 H) 5.92 (d, J=17.7 Hz, 1 H) 5.39 (d, J=11.1 Hz, 1 H) 3.83-4.01 (m, 4 H) 3.69-3.83 (m, 4 H). MS(ES+) m/e 242 [M+H]⁺.
d) 3-(4-Morpholinyl)-6-quinoxalinecarbaldehyde. A solution of the compound from Example 1 c) (0.464 g; 1.92 mmol) in dioxane (6.0 mL) and water (2.0 mL) was treated with osmium tetroxide (0.482 mL; 2.5% soln. in t-BuOH; 0.038 mmol), 2,6-lutidine (0.448 mL; 3.85 mmol), and sodium periodate (0.825 g; 3.85 mmol). After stirring 30 min. at ambient temperature, the reaction was quenched with water, extracted thrice with CH₂Cl₂, dried over magnesium sulfate, filtered, and concentrated in vacuo. Purification via flash column chromatography (10-60% ethyl acetate in hexanes) provided the title compound as a light yellow solid (0.425 g, 91%). 1H NMR (400 MHz, CHLOROFORM-d) δ ppm 10.16 (s, 1 H) 8.65 (s, 1 H) 8.16 (d, J=1.5 Hz, 1 H) 7.98 (d, J=8.4 Hz, 1 H) 7.89 (dd, J=8.4, 1.8 Hz, 1 H) 3.85-3.93 (m, 4 H) 3.78-3.85 (m, 4 H). MS(ES+) m/e 244 [M+H]⁺.
e) (5Z)-2-[(2,6-Dichlorophenyl)amino]-5-{[3-(4-morpholinyl )-6-quinoxalinyl]methylidene}-1,3-thiazol-4(5H)-one. A solution of the compound from Example 1d) (0.263 g; 1.01 mmol), 2-[(2,6-dichlorophenyl)amino]-1,3-thiazol-4(5H)-one (0.245 g; 1.01 mmol) and piperidine (0.100 mL; 1.01 mmol) in ethanol (2.0 mL) was stirred and heated at 150° C. for 30 min. in a Biotage Initiator microwave synthesizer. The reaction mixture was then cooled, poured into 1M aqueous hydrochloric acid (5.0 mL), filtered, and washed with water to give the title compound as a brown solid (0.421 g; 86%). 1H NMR (400 MHz, CHLOROFORM-d) δ ppm 8.55 (s, 1 H) 7.88 (d, J=8.6 Hz, 1 H) 7.77 (s, 1 H) 7.72 (d, J=0.8 Hz, 1 H) 7.47 (dd, J=8.6, 1.3 Hz, 1 H) 7.39 (d, J=8.1 Hz, 2 H) 7.07 (t, J=8.1 Hz, 1 H) 4.78 (s, 1 H) 3.82-3.97 (m, 4 H) 3.74-3.81 (m, 4 H). MS(ES+) m/e 486 [M+H]⁺.

### EXAMPLE 2

7-{(Z)-[2-[(2,6-Dichlorophenyl)amino]-4-oxo-1,3-thiazol-5(4H )-ylidene]methyl}-2(1H)-quinoxalinone
a) 7-Ethenyl-2(1H)-quinoxalinone. A solution of 7-bromo-2(1H)-quinoxalinone (prepared by the method of Linda, P; Marino, G. Ric. Sci., Rend. Sez. A. [2] 1963, 3, 225-228)(1.04 g; 6.22 mmol), tributyl(vinyl)tin (2.0 mL; 6.84 mmol), and tetrakis(triphenylphosphine)palladium(0) (0.072 g; 0.062 mmol) in dioxane (10.0 mL) was stirred and heated to 150° C. for 20 min. in a Biotage Initiator microwave synthesizer. Concentration in vacuo and purification via flash column chromatography (20-50% ethyl acetate in hexanes) provided the title compound as a yellow solid (1.05 g, 98%). 1H NMR (400 MHz, DMSO-d₆) δ ppm 12.43 (s, 1 H) 8.12 (s, 1 H) 7.74 (d, J=8.3 Hz, 1 H) 7.48 (dd, J=8.3, 1.8 Hz, 1 H) 7.28 (d, J=1.8 Hz, 1 H) 6.84 (dd, J=17.7, 10.9 Hz, 1 H) 5.94 (d, J=17.7 Hz, 1 H) 5.43 (d, J=11.4 Hz, 1 H). MS(ES+) m/e 173 [M+H]⁺.
b) 3-Oxo-3,4-dihydro-6-quinoxalinecarbaldehyde. A solution of the compound from Example 2a) (0.075 g; 0.436 mmol) in dioxane (3.0 mL) and water (1.0 mL) was treated with osmium tetroxide (0.109 mL; 2.5% soln. in t-BuOH; 0.009 mmol), 2,6-lutidine (0.101 mL; 0.871 mmol), and sodium periodate (0.186 g; 0.871 mmol). After stirring 30 min. at ambient temperature, the reaction was quenched with water, extracted thrice with CH₂Cl₂, dried over magnesium sulfate, filtered, and concentrated in vacuo to give the title compound as a light yellow solid (0.074 g, 98%). 1H NMR (400 MHz, DMSO-d₆) δ ppm 12.70 (s, 1 H) 10.09 (s, 1 H) 8.31 (d, J=2.3 Hz, 1 H) 7.98 (d, J=8.1 Hz, 1 H) 7.80 (dd, J=8.1, 1.5 Hz, 1 H) 7.79 (s, 1 H). MS(ES+) m/e 175 [M+H]⁺.
c) 7-{(Z)-[2-[(2,6-Dichlorophenyl)amino]-4-oxo-1,3-thiazol-5(4H)-ylidene]methyl}-2(1H)-quinoxalinone. A solution of the compound from Example 2b) (0.067 g; 0.383 mmol), 2-[(2,6-dichlorophenyl)amino]-1,3-thiazol-4(5H)-one (0.100 g; 0.383 mmol) and piperidine (0.038 mL; 0.383 mmol) in ethanol (2.0 mL) was stirred and heated at 150° C. for 30 min. in a Biotage Initiator microwave synthesizer. The reaction mixture was then cooled, poured into 1M aqueous hydrochloric acid (5.0 mL), filtered, and washed with water to give the title compound as an orange solid (0.129 g; 81%). 1H NMR (400 MHz, DMSO-d₆) δ ppm 13.05 (s, 1 H) 12.45 (s, 1 H) 8.18 (d, J=2.3 Hz, 1 H) 7.83 (d, J=8.3 Hz, 1 H) 7.76 (s, 1 H) 7.58 (d, J=8.1 Hz, 2 H) 7.47 (dd, J=8.3, 1.5 Hz, 1 H) 7.37 (d, J=0.8 Hz, 1 H) 7.24 (t, J=8.1 Hz, 1 H). MS(ES+) m/e 417 [M+H]⁺.

### EXAMPLE 3

(5Z)-2-[(2,6-Dichlorophenyl)amino]-5-{[2-(methylamino)-6-quinolinyl]methylidene}-1,3-thiazol-4(5H)-one
a) (5Z)-5-[(2-Chloro-6-quinolinyl)methylidene]-2-[(2,6-dichlorophenyl)amino]-1,3-thiazol-4(5H)-one. A solution of 2-chloro-6-quinolinecarbaldehyde (S. Inglis et. al., J. Med. Chem., 2004, 47(22), 5405; 20 mg, 0.1 mmol), 2-[(2,6-dichlorophenyl)amino]-1,3-thiazol-4(5H)-one (26 mg, 0.1 mmol) and piperazine (0.010 mL, 0.1 mmol) in ethanol (2.0 mL) was stirred and heated at 150° C. for 30 min. in a Biotage Initiator microwave synthesizer. The reaction mixture was then cooled, poured into 1M aqueous hydrochloric acid (5.0 mL), filtered, and washed with water to give the title compound as a yellow solid (18 mg, 40%). MS(ES+) m/e 433[M+H]⁺. 1H NMR (400 MHz, CHLOROFORM-d) δ ppm 8.15 (d, J=8.84 Hz, 1 H) 8.04 (d, J=8.84 Hz, 1 H) 7.89 (s, 1 H) 7.87 (s, 1 H) 7.76 (dd, J=8.84, 1.77 Hz, 1 H) 7.38-7.46 (m, 1 H) 7.12 (t, J=8.08 Hz, 1 H).
b) (5Z)-2-[(2,6-Dichlorophenyl)amino]-5-{[2-(methylamino)-6-quinolinyl]methylidene}-1,3-thiazol-4(5H)-one. To a solution of compound from Example 3a) (10 mg, 0.023 mmol) in MeOH (2 mL) was added CH₃NH₂ (2.0 M in MeOH, 2 mL, 4 mmol). The mixture was refluxed for 5 days, cooled and purified by HPLC (YMC HPLC column 75×30 mml.D. 0-100% acetonitrile (0.1% TFA) in water (0.1% TFA)) to give the title compound as yellow solid (4 mg, 41%). MS(ES+) m/e 430 [M+H]⁺. 1H NMR (400 MHz, DMSO-d₆) δ ppm 13.01 (s, 1 H) 9.82 (s, 1 H) 8.29 (d, J=9.35 Hz, 1 H) 7.95-8.07 (m, 2 H) 7.83 (d, J=8.59 Hz, 1 H) 7.80 (s, 1 H) 7.57 (d, J=8.08 Hz, 2 H) 7.24 (t, J=8.21 Hz, 1 H) 7.05 (d, J=9.09 Hz, 1 H) 3.10 (d, J=4.29 Hz, 3 H).

### EXAMPLE 4

(5Z)-2-[(2,6-Dichlorophenyl)amino]-5-{[4-(dimethylamino)-6-quinolinyl]methylidene}-1,3-thiazol-4(5H)-one
a) 6-Bromo-N,N-dimethyl-4-quinolinamine. To a solution of 6-bromo-4-chloroquinoline (200 mg, 0.41 mmol) (prepared by the method of Ai Jeng Lin, J. Med. Chem, 1978, 21, 268) in dry MeOH (2.0 mL) was added dimethylamine hydrochloride (67 mg, 0.82 mmol) and N,N-diisopropylethylamine (0.14 mL, 0.82 mmol). The mixture was heated at 150° C. in a Biotage Initiator microwave synthesizer for 20 minutes, then cooled and quenched with H₂O. The resulting mixture was extracted by CH₂Cl₂. The extract was dried over MgSO₄, filtered, concentrated in vacuo and purified by flash chromatography ( 0-10% MeOH in CH₂Cl₂ ) to afford colorless oil (130 mg, 62%). MS(ES+) m/e 252[M+H]⁺. 1H NMR (400 MHz, CHLOROFORM-d) δ ppm 8.61 (d, J=4.29 Hz, 1 H) 8.17 (d, J=2.02 Hz, 1 H) 7.87 (d, J=8.84 Hz, 1 H) 7.66 (dd, J=8.97, 2.15 Hz, 1 H) 6.71 (d, J=5.31 Hz, 1 H) 2.93-3.02 (m, 6 H).
b) 4-(Dimethylamino)-6-quinolinecarbaldehyde. To a solution of compound from Example 4a) (130 mg, 0.52 mmol) in dry THF at −78° C. was added n-BuLi (1.6 M in hexane, 0.32 mL, 0.52 mmol), then DMF (0.080 mL, 1.04 mmol). The mixture was warmed up to the room temperature, quenched by saturated NH₄Cl and extracted by ethyl acetate. The extract was dried over MgSO₄, filtered and concentrated in vacue. The residue was purified by flash chromatography (0-100% ethyl acetate in hexanes) to afford brown solid (40 mg, 40%). MS(ES+) m/e 201[M+H]⁺.
c) (5Z)-2-[(2,6-dichlorophenyl)amino]-5-{[4-(dimethylamino)-6-quinolinyl]methylidene}-1,3-thiazol-4(5H)-one. To a solution of compound from Example 4b) (37.2 mg, 0.2 mmol) in ethanol (2 mL) was added 2-[(2,6-dichlorophenyl)amino]-1,3-thiazol-4(5H)-one (52 mg, 0.2 mmol) and piperidine ( 0.040 mL, 0.4 mmol). The mixture was stirred and heated at 150° C. for 30 min. in a Biotage Initiator microwave synthesizer. The solvent was removed in vacue. The residue was purified by flash chromatography (0-10% MeOH in CH₂Cl₂) to afford the title compound as yellow solid (25 mg, 38%). MS(ES+) m/e 443 [M+H]⁺. 1H NMR (400 MHz, DMSO-d₆)δ ppm 8.54 (d, J=5.81 Hz, 1 H) 8.29-8.38 (m, 1 H) 7.90-8.02 (m, 2 H) 7.78 (d, J=8.59 Hz, 1 H) 7.58 (d, J=8.08 Hz, 2 H) 7.24 (t, J=8.08 Hz, 1 H) 6.90 (d, J=5.81 Hz, 1 H) 3.15 (s, 6 H).

### EXAMPLE 5

(5Z)-2-[(2,6-Dichlorophenyl)amino]-5-{[4-(4-methyl-1-piperazinyl)-6-quinolinyl]methylidene}-1,3-thiazol-4(5H)-one
a) 4-Chloro-6-ethenylquinoline. A solution of the 6-bromo-4-chloroquinoline (4g; 16.5 mmol), tributyl(vinyl)tin (4.8 ml, 16.5 mmol), and tetrakis(triphenylphosphine)palladium(0) (0.190 g; 0.16 mmol) in dioxane (15.0 mL) was stirred and heated to 150° C. for 20 min. in a Biotage Initiator microwave synthesizer. Concentration in vacuo and purification via flash column chromatography (0-100% ethyl acetate in hexanes) provided the title compound as a yellow solid (2.5 g, 80%). MS(ES+) m/e 190 [M+H]⁺. 1H NMR (400 MHz, CHLOROFORM-d) δ ppm 8.72 (d, J=4.55 Hz, 1 H) 8.02-8.13 (m, 2 H) 7.91 (dd, J=8.72, 1.89 Hz, 1 H) 7.47 (d, J=4.80 Hz, 1 H) 6.93 (dd, J=17.68, 10.86 Hz, 1 H) 5.95 (d, J=17.43 Hz, 1 H) 5.45 (d, J=11.12 Hz, 1 H).
b) 4—Chloro-6-quinolinecarbaldehyde. A solution of the compound from Example 5a) (5.36 g, 28.35 mmol) in dioxane (15.0 mL) and water (5.0 mL) was treated with osmium tetroxide (7.1 mL, 2.5% soln. in t-BuOH, 0.57 mmol), 2,6-lutidine (6.6 mL, 56.7 mmol), and sodium periodate (24.3 g, 113.4 mmol). After stirring 2 hours at ambient temperature, the reaction was quenched with water, extracted twice with CH₂Cl₂, dried over magnesium sulfate, filtered, and concentrated in vacuo. Purification via flash column chromatography (0-100% ethyl acetate in hexanes) provided the title compound as a white solid (4.5 g, 84%). MS(ES+) m/e 192 [M+H]⁺. 1H NMR (400 MHz, CHLOROFORM-d) δ ppm 10.27 (s, 1 H) 8.93 (d, J=4.55 Hz, 1 H) 8.77 (s, 1 H) 8.19-8.34 (m, 2 H) 7.62 (d, J=4.80 Hz, 1 H).
c) (5Z)-2-[(2,6-Dichlorophenyl)amino]-5-{[4-(4-methyl-1-piperazinyl)-6-quinolinyl]methylidene}-1,3-thiazol-4(5H)-one hydrochloride. A solution of the compound from Example 5b) (200 mg, 1.05 mmol), 2-[(2,6-dichlorophenyl)amino]-1,3-thiazol-4(5H)-one (272 mg, 1.05 mmol) and 1-methylpiperazine (0.23 mL, 2.1 mmol) in ethanol (2.0 mL) was stirred and heated at 150° C. for 30 min. in a Biotage Initiator microwave synthesizer. The reaction mixture was then cooled, poured into 1M aqueous hydrochloric acid (5.0 mL), filtered, and washed with water to give the title compound as a yellow solid (0.34 g; 65%). MS(ES+) m/e 498 [M+H]⁺. 1H NMR (400 MHz, DMSO-d₆) δ ppm 13.10 (s, 1 H) 12.00 (s, 1 H) 8.80 (s, 1 H) 8.31 (s, 1 H) 8.22 (s, 1 H) 8.05 (s, 1 H) 7.97 (s, 1 H) 7.55 (d, J=1.52 Hz, 2 H) 7.35 (s, 1 H) 7.21 (s, 1 H) 4.24 (s, 2 H) 3.86 (s, 2 H) 3.16-3.54 (m, 4 H) 2.79 (s, 3 H).

### EXAMPLE 6—Capsule Composition

An oral dosage form for administering the present invention is produced by filing a standard two piece hard gelatin capsule with the ingredients in the proportions shown in Table I, below.

TABLE I

| INGREDIENTS | AMOUNTS |
|---|---|
| (5Z)-2-[(2,6-Dichlorophenyl)amino]-5-{[3-(4- | 25 mg |
| morpholinyl)-6-quinoxalinyl]methylidene}-1,3- | |
| thiazol-4(5H)-one | |
| Lactose | 55 mg |
| Talc | 16 mg |
| Magnesium Stearate | 4 mg |

### Example 7—Injectable Parenteral Composition

An injectable form for administering the present invention is produced by stirring 1.5% by weight of 7-{(Z)-[2-[(2,6-Dichlorophenyl)amino]-4-oxo-1,3-thiazol-5(4H)-ylidene]methyl}-2(1H)-quinoxalinone in 10% by volume propylene glycol in water.

### EXAMPLE 8—Tablet Composition

The sucrose, calcium sulfate dihydrate and an hYAK inhibitor as shown in Table II below, are mixed and granulated in the proportions shown with a 10% gelatin solution. The wet granules are screened, dried, mixed with the starch, talc and stearic acid; screened and compressed into a tablet.

TABLE II

| INGREDIENTS | AMOUNTS | |
|---|---|---|
| (5Z)-2-[(2,6-Dichlorophenyl)amino]-5-{[2- | 20 | mg |
| (methylamino)-6-quinolinyl]methylidene}-1,3- | | |
| thiazol-4(5H)-one | | |
| calcium sulfate dihydrate | 30 | mg |
| sucrose | 4 | mg |
| starch | 2 | mg |
| talc | 1 | mg |
| stearic acid | 0.5 | mg |

While the suitable embodiments of the invention are illustrated by the above, it is to be understood that the invention is not limited to the precise instructions herein disclosed and that the right to all modifications coming within the scope of the following claims is reserved.

What is claimed is:

1. A method of inhibiting one or more phosphatoinositides 3-kinases (PI3Ks) in a mammal; comprising administering to the mammal a therapeutically effective amount of a compound of Formula (I): 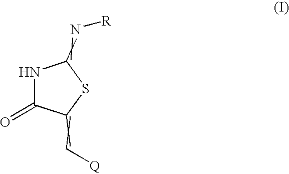
in which
R is selected form: aryl and substituted aryl; and
Q is 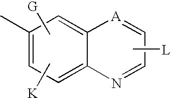
wherein
A is selected from CR⁵⁰ and N, where R⁵⁰, G, K and L are each independently selected from the group consisting of: hydrogen, amino, alkylamine, substituted alkylamine, dialkylamine, substituted dialkylamine, hydroxy, alkylaminoalkyl, dialkylaminoalkyl, alkoxy, alkyl, substituted alkyl, aryl, substituted aryl, arylamine, substituted arylamine, halogen, cycloalkyl, substituted cycloalkyl, cycloalkyl containing from 1 to 4 heteroatoms, substituted cycloalkyl containing from 1 to 4 heteroatoms, —C(O)OR¹⁰, —C(O)NR¹¹R¹² and cyano,
where, R¹⁰ is selected form hydrogen, C₁-C₄ alkyl, aryl and trifluoromethyl, and R¹¹ and R¹² are independently selected form hydrogen, C₁-C₄ alkyl, aryl and trifluoromethyl,
provided that at least one of G, K, L and R⁵⁰, when R⁵⁰ is present, is not hydrogen,
and/or a pharmaceutically acceptable salt, hydrate, solvate or pro-drug thereof.

2. A method of treating one or more disease state selected from the group consisting of: autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries, in a mammal, which method comprises administering to such mammal, a therapeutically effective amount of a compound according to claim 1.

3. A method of treating cancer comprises co-administration a compound of formula I and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

4. The method of claim 3, wherein the disease state is selected from the group consisting of: multiple sclerosis, psoriasis, rheumatoid arthritis, systemic lupus erythematosis, inflammatory bowel disease, lung inflammation, thrombosis, brain infection/inflammation, meningitis and encephalitis.

5. The method of claim 3, wherein the disease state is selected from the group consisting of: Alzheimer's disease, Huntington's disease, CNS trauma, stroke and ischemic conditions.

6. The method of claim 3, wherein the disease state is selected from the group consisting of: atherosclerosis, heart hypertrophy, cardiac myocyte dysfunction, elevated blood pressure and vasoconstriction.

7. The method of claim 3, wherein the disease state is selected from the group consisting of: chronic obstructive pulmonary disease, anaphylactic shock fibrosis, psoriasis, allergic diseases, asthma, stroke, ischemia-reperfusion, platelets aggregation/activation, skeletal muscle atrophy/hypertrophy, leukocyte recruitment in cancer tissue, antiogenesis, invasion metastasis, melanoma, Karposi's sarcoma, acute and chronic bacterial and virual infections, sepsis, transplantation rejection, graft rejection, glomerulo sclerosis, glomerulo nephritis, progressive renal fibrosis, endothelial and epithelial injuries in the lung, and lung airways inflammation.

8. The method of claim 3 wherein the disease is cancer.

9. The method of claim 3 wherein the disease is selected from a group consisting of: ovarian cancer, pancreatic cancer, breast cancer, prostate cancer and leukemia.

10. The method of claim 3 wherein the mammal is human.

11. The method of claim 1, wherein said PI3 kinase is a PI3α.

12. The method of claim 1, wherein said PI3 kinase is a PI3γ.

13. The method of claim 1, wherein said compound is selected from:
(5Z)-2-[(2,6-Dichlorophenyl)amino]-5-{[3-(4-morpholinyl)-6-quinoxalinyl]methylidene}-1,3-thiazol-4(5H)-one;
7-{(Z)-[2-[(2,6-Dichlorophenyl)amino]-4-oxo-1,3-thiazol-5(4H)-ylidene]methyl}-2(1H)-quinoxalinone;
(5Z)-2-[(2,6-Dichlorophenyl)amino]-5-{[2-(methylamino)-6-quinolinyl]methylidene}-1,3-thiazol-4(5H)-one;
(5Z)-2-[(2,6-Dichlorophenyl)amino]-5-{[4-(dimethylamino)-6-quinolinyl]methylidene}-1,3-thiazol-4(5H)-one; and
(5Z)-2-[(2,6-Dichlorophenyl)amino]-5-{[4-(4-methyl-1-piperazinyl)-6-quinolinyl]methylidene}-1,3-thiazol-4(5H)-one.

14. A method of claim 1 wherein the compound of formula (I), and/or a pharmaceutically acceptable salt, hydrate, solvate or pro-drug thereof, is administered in a pharmaceutical composition.
